(19) United States
(12) Patent Application Publication
(10) Pub. No.: US 2002/0006933 A1
(43) Pub. Date: Jan. 17, 2002

(54) Method for treating sexual dysfunction with apomorphine at specified plasma concentration levels

(75) Inventors: Pramod K. Gupta, Gurnee, IL (US); John Daniel Bollinger, Libertyville, IL (US); Yisheng Chen, Gurnee, IL (US); Jack Yuqun Zheng, Lake Bluff, IL (US); Thomas L. Reiland, Gages Lake, IL (US); Dennis Y. Lee, Highland Park, IL (US)

Correspondence Address: ROCKEY, MILNAMOW & KATZ, LTD., TWO PRUDENTIAL PLAZA, STE. 4700, 180 NORTH STETSON AVENUE, CHICAGO, IL, 60601, US

(73) Assignee: TAP Holdings, Inc.

(21) Appl. No.: 09/808,605

(22) Filed: Mar. 14, 2001

Related U.S. Application Data
(60) Provisional application No. 60/190,540, filed on Mar. 20, 2000.

(51) Int. Cl. A61K 31/4748; A61K 9/00

(52) U.S. Cl. 514/295; 424/43

(57) ABSTRACT

Methods for administering apomorphine to a patient for the treatment of sexual dysfunctions while reducing undesirable side effects are disclosed. In the methods, the concentration of apomorphine is attained within the patients' plasma of up to 10 nanograms per milliliter. Advantageously, this concentration may be achieved with less than 15% of patients so treated experiencing emesis. Methods of administration are intranasally, by inhalation to the lungs or by oral ingestion.

### FIELD OF THE INVENTION

The present invention is directed to a method for administering apomorphine to a patient for the treatment of sexual dysfunction while reducing undesirable side effects. In the method, the concentration of apomorphine is attained within the patients' plasma of up to 10 nanograms per milliliter. Advantageously, this concentration may be achieved with less than 15% of patients so treated experiencing emesis. Methods of administration are intranasally, by inhalation to the lungs or by oral ingestion.

### BACKGROUND OF THE INVENTION

The human sexual response in both males and females results from a complex interplay of psychological, hormonal and other physiological influences. Efforts are ongoing to provide effective treatments which are convenient and simple to use, do not require a constant dosage regimen or even multiple doses to achieve desired results, are non-invasive and allow a rapid and predictable capacity for sexual function on demand and in response to normal sexual stimulation.

For males, methods involving various external devices for the treatment of impotence have been suggested such as tourniquets (see U.S. Pat. No. 2,818,855). In addition, penile implants, such as hinged or solid rods and inflatable, spring driven or hydraulic models, have been used for some time.

Drug treatments are also known. For example, U.S. Pat. No. 4,127,118 discloses a method of treating male impotence by local injection of an appropriate vasodilator, in particular, an adrenergic blocking agent or a smooth muscle relaxant to effect and enhance an erection, and U.S. Pat. No. 4,801,587 discloses the application of an ointment to relieve impotence. The ointment consists of the vasodilators papaverine, hydralazine, sodium nitroprusside, phenoxybenzamine, or phentolamine and a carrier to assist absorption of the primary agent through the skin. U.S. Pat. No. 5,256,652 discloses the use of an aqueous topical composition of a vasodilator such as papaverine together with hydroxypropyl-β-cyclodextrin.

The effect of apomorphine on impotence, or male sexual dysfunction has been extensively studied and reported upon. However, apomorphine has been shown to have very poor oral bioavailability. See, for example, Baldessarini et al., in Gessa et al., eds., Apomorphine and Other Dopaminomimetics, Basic Pharmacology, Vol. 1, Raven Press, New York (1981), pp. 219-228.

Therefore, the efficacy of the use of apomorphine for treatment of sexual dysfunction is reduced by the problems of low bioavailability and undesirable side effects. An increased bioavailability leads to an increase in plasma concentration of the drug and an increase in undesirable side effects. Therefore, for the treatment of sexual dysfunction, use of apomorphine has to date been qualified by specific concentration parameters and/or methods of administration to overcome this problem.

For example, apomorphine has been disclosed for the amelioration of female sexual dysfunction in U.S. Pat. No. 5,945,117. Apomorphine has also been disclosed for the amelioration of male erectile dysfunction in U.S. Pat. Nos. 5,624,677; 5,888,534; 5,770,606; 5,985,889 and 5,994,363. In U.S. Pat. No. 5,624,677, mint flavoring may be added to the formulation to attenuate some of the local emesis receptors. In U.S. Pat. No. 5,888,534, a slow release sublingual tablet is disclosed. The slow release of the tablet is said to reduce the undesirable side effects of the drug. The adverse effects of apomorphine were minimized by gradual acclimatization to apomorphine as disclosed in U.S. Pat. No. 5,994,363. Apomorphine was disclosed for treatment of impotence in a fast release oral formulation when the patient was first pre-treated with domperidone in WO 98/31368. The treatment of erectile dysfunction with certain nasal formulations of apomorphine is disclosed in WO 99/27905.

In U.S. Pat. Nos. 5,770,606 and 5,985,889 sublingual administration of apomorphine such that a plasma concentration of no more than 5.5 ng/ml was maintained was disclosed to alleviate undesirable side effects. Moreover, the '889 patent indicates that though apomorphine was evaluated as an aqueous intranasal spray in Pilot Study #3, one patient's highly adverse reaction led to discontinuation of further testing and a recognition that there is still a need for reliable and relatively safe dosage formulations.

Therefore, there is a need for alternative methods of administration of apomorphine which provide the requisite bioavailability, while minimizing undesirable side effects.

We have now discovered that other routes of administration may provide a higher bioavailability than the bioavailability obtained from conventional sublingual treatment and yet do not result in a proportional increase in undesirable side effects contrary to principles understood by those skilled in the art.

### SUMMARY OF THE INVENTION

The present invention is directed to methods for administering apomorphine to a patient for the treatment of sexual dysfunctions while reducing undesirable side effects. In the methods, apomorphine is maintained at a concentration within the patients' plasma of up to 10 nanograms per milliliter. More particularly, the present invention is directed to a method of treating sexual dysfunction in a patient comprising

administering a therapeutically effective amount of apomorphine or a pharmaceutically acceptable salt thereof to said patient intranasally, by inhalation to the lungs or by oral ingestion;

wherein a concentration of said apomorphine is attained within said patient's plasma of up to 10 nanograms per milliliter;

and wherein said concentration is achieved with less than 15% of patients so treated experiencing emesis.

The present invention is also directed to a method of treating sexual dysfunction in a patient comprising

administering a therapeutically effective amount of apomorphine or a pharmaceutically acceptable salt thereof to said patient;

wherein a concentration of apomorphine is attained within said patient's plasma of up to 10 nanograms per milliliter;

and wherein said concentration is achieved with less than 15% of patients so treated experiencing emesis;

with the proviso that administration is not sublingual.

The apomorphine may be administered intranasally, by inhalation to the lungs, or by oral ingestion.

Intranasal administration may be accomplished by the use of a nasal spray, nasal drops, gel, suspension, ointment, cream or powder.

“Ingested orally” or “oral ingestion” as used herein indicate that the drug will primarily be pushed beyond the mouth to the stomach; so that the mouth is the point of entry but not the primary point of absorption. Thus, the terms “ingested orally” or “oral ingestion” as used herein are meant to distinguish a primarily oral absorption wherein the mouth is the point of entry and absorption occurs primarily in the stomach, from oral-mucosal administration wherein the mouth is both the point of entry and the point of absorption, or oral administration of fast dissolving tablets wherein the mouth is the point of entry but the mouth and mucosal membranes are the point of absorption. The apomorphine may be orally ingested in the form of a solution, suspension, drops, a gel, a tablet, granules, sprinkles, pills, powder or a capsule.

For the practice of any of the methods of this invention, the sexual dysfunction may be erectile dysfunction. The concentration may be attained without substantial adverse effects, such as emesis. Specifically, the concentration may be achieved with less than 15% of patients so treated experiencing emesis. The method for treating sexual dysfunction may be utilized to treat either males or females. For the practice of any of the methods of the present invention, the plasma concentration of apomorphine may preferably be from about 0.1 to about 7 ng/ml. Presently most preferably, the plasma concentration of apomorphine may be from about 0.5 to about 5 ng/ml.

The present invention is also directed to a method of treating sexual dysfunction in a patient comprising

intranasally administering a therapeutically effective amount of apomorphine or a pharmaceutically acceptable salt thereof to said patient;

wherein a concentration of apomorphine is attained within said patient's plasma of up to 10 nanograms per milliliter.

For the intranasal route, the apomorphine may be administered as a nasal spray, nasal drops, gel, suspension, ointment, cream or powder.

The present invention is also directed to a method of treating sexual dysfunction in a patient comprising

administering a therapeutically effective amount of apomorphine or a pharmaceutically acceptable salt thereof to said patient by oral ingestion;

wherein a concentration of apomorphine is attained within said patient's plasma of up to 10 nanograms per milliliter.

For oral ingestion, the apomorphine may be administered as a solution, a suspension, drops, a gel, a tablet, pills, powder, granules, sprinkles or a capsule.

The present invention is also directed to a method of treating sexual dysfunction in a patient comprising

administering a therapeutically effective amount of apomorphine or a pharmaceutically acceptable salt thereof by inhalation to the lungs of said patient;

wherein a concentration of apomorphine is attained within said patient's plasma of up to 10 nanograms per milliliter.

The delivery device or the method of administration for inhalation may include metered dose inhalers, dry powder inhalers, nebulization of a solution or suspension and/or any other system which achieves the same results.

### DETAILED DESCRIPTION OF THE INVENTION

In males, the form of sexual dysfunction is erectile dysfunction. A normal erection occurs as a result of a coordinated vascular event in the penis. This is usually triggered neurally and consists of vasodilation and smooth muscle relaxation in the penis and its supplying arterial vessels. Arterial inflow causes enlargement of the substance of the corpora cavernosa. Venous outflow is trapped by this enlargement, permitting sustained high blood pressures in the penis sufficient to cause rigidity. Muscles in the perineum also assist in creating and maintaining penile rigidity. Erection may be induced centrally in the nervous system by sexual thoughts or fantasy, and is usually reinforced locally by reflex mechanisms. Erectile mechanics are substantially similar in the female for the clitoris.

Impotence or male erectile dysfunction is defined as the inability to achieve and sustain an erection sufficient for intercourse. Impotence in any given case can result from psychological disturbances (psychogenic), from physiological abnormalities in general (organic), from neurological disturbances (neurogenic), hormonal deficiencies (endocrine) or from a combination of the foregoing. Impotence may be hormonal, congenital, vascular or partial ability, among others.

These descriptions are not exact, however. There is currently no standardized method of diagnosis or treatment. As used herein, psychogenic impotence is defined as functional impotence with no apparent overwhelming organic basis. It may be characterized by an inability to have an erection in response to some stimuli (e.g., masturbation, spontaneous nocturnal, spontaneous early morning, video erotica, etc.) but not others (e.g., partner or spousal attention).

Females also can have sexual dysfunction that increases with age and is associated with the presence of vascular risk factors and onset of menopause. Some of the vascular and muscular mechanisms that contribute to penile erection in the male are believed to be similar vasculogenic factors in female genital response. It is known that in women, sexual arousal is accompanied by arterial inflow which engorges the vagina and increases vaginal lubrication and that the muscles in the perineum assist in achieving clitoral erection.

In the female, sexual dysfunction can arise from organic and psychogenic causes or from a combination of the foregoing. Female sexual dysfunction includes a failure to attain or maintain vaginal lubrication-swelling responses of sexual excitement until completion of the sexual activity. Organic female sexual dysfunction is known to be related in part to vasculogenic impairment resulting in inadequate blood flow, vaginal engorgement insufficiency and clitoral erection insufficiency.

Apomorphine ((R)-5,6,6a,7-tetrahydro-6-methyl-4H-dibenzo-[de,g]quinoline-10,11-diol) can be represented by the formula

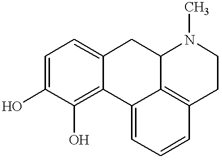

and exists in a free base form or as an acid addition salt. For the purposes of the present invention, apomorphine hydrochloride is preferred, however other pharmacologically acceptable moieties forms of apomorphine can be utilized as well.

Apomorphine can be used in the form of pharmaceutically acceptable salts derived from inorganic or organic acids . The phrase “pharmaceutically acceptable salt” means those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66: 1 et seq. The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzene sulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluene sulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Apomorphine has been disclosed as useful in intranasal formulations for the treatment of Parkinson's disease in U.S. Pat. No. 5,756,483. Apomorphine transdermal administration has been disclosed in U.S. Pat. No. 5,939,094; and apomorphine in capsule form has been disclosed in U.S. Pat. No. 5,866,164.

Apomorphine is a dopamine receptor agonist that has a recognized use as an emetic when administered subcutaneously in about a 5 milligram dose. For the purposes of the present invention, apomorphine or a similarly acting dopamine receptor agonist is administered in an amount sufficient to excite cells in the mid-brain region of the patient but with minimal side effects. This cell excitation is believed to be part of a cascade of stimulation that is likely to include neurotransmission with serotonin, dopamine and oxytocin.

Apomorphine according to the invention can be administered as a nasal spray, nasal drop, suspension, gel, ointment, cream or powder. The administration of the nasal composition may also take place using a nasal tampon or nasal sponge.

Powders can be administered using a nasal insufflator. Powders can also be used in such a manner that they are placed in a capsule. The capsule is set in an inhalation or insufflation device. A needle is penetrated through the capsule to make pores at the top and the bottom of the capsule, and air is sent to blow out the powder particles. Powder formulations can also be administered in a jet-spray of an inert gas or suspended in liquid organic fluids.

The present invention provides a method for the treatment of sexual dysfunction with a pharmaceutical composition comprising apomorphine and pharmaceutically acceptable salts thereof and a physiologically tolerable diluent. The present invention includes apomorphine and pharmaceutically acceptable salts thereof formulated into compositions together with one or more non-toxic physiologically tolerable or acceptable diluents, carriers, adjuvants or vehicles that are collectively referred to herein as diluents, for intranasal delivery or for oral administration in solid or liquid form.

These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Useful intranasal formulations contain a stabilizer and a surfactant. Among the pharmaceutically acceptable surfactants are polyoxyethylene castor oil derivatives, such as polyoxyethylene-glycerol-triricinoleate, also known as polyoxyl 35 castor oil (CREMOPHOR EL), or poloxyl 40 hydrogenated castor oil (CREMOPHOR RH40) both available from BASF Corp.; mono-fatty acid esters of polyoxyethylene (20) sorbitan, such as polyoxyethylene (20) sorbitan monolaurate (TWEEN 80), polyoxyethylene monostearate (TWEEN 60), polyoxyethylene (20) sorbitan monopalmitate (TWEEN 40), or polyoxyethylene 20 sorbitan monolaurate (TWEEN 20) all available from ICI Surfactants of Wilmington, Del.); polyglyceryl esters, such as polyglyceryl oleate; and polyoxyethylated kernel oil (LABRAFIL, available from Gattefosse Corp.) Preferably, the surfactant will be between about 0.01% and 10% by weight of the pharmaceutical composition.

Among the pharmaceutically useful stabilizers are antioxidants such as sodium sulfite, sodium metabisulfite, sodium thiosulfate, sodium formaldehyde sulfoxylate, sulfur dioxide, ascorbic acid, isoascorbic acid, thioglycerol, thioglycolic acid, cysteine hydrochloride, acetyl cysteine, ascorbyl palmitate, hydroquinone, propyl gallate, nordihydroguaiaretic acid, butylated hydroxytoluene, butylated hydroxyanisole, alpha-tocopherol and lecithin. Preferably, the stabilizer will be between about 0.01% and 5% by weight of the pharmaceutical composition.

Chelating agents such as ethylene diamine tetraacetic acid, its derivatives and salts thereof, dihydroxyethyl glycine, citric acid and tartaric acid among others may also be utilized.

Proper fluidity can be maintained, for example, by the use of coating materials such as lecithin, by the maintenance of the required particle size in the case of dispersions and by the use of surfactants.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

The drug can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to the drug, stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology, Volume XIV, Academic Press, New York, N.Y. (1976), p. 33 et seq.

The moderation of undesirable side effects of apomorphine depending upon the route of administration or formulation of the drug is described in detail hereinafter in the Examples. These Examples are presented to describe preferred embodiments and utilities of the invention and are not meant to limit the invention unless otherwise stated in the claims appended hereto.

### EXAMPLE 1

The moderation of undesirable side effects of apomorphine when administered intranasally as compared to the conventional sublingual route was studied in dogs. Dogs have been shown to be an appropriate model for study as disclosed in U.S. Pat. No. 5,994,363 Example 3. The bioavailability of apomorphine sub-lingual tablets in dogs have been shown to be comparable to the bioavailability by the same route of administration in humans. Dogs are known to be 5 to 10 times more sensitive than humans to apomorphine-induced emesis.

The drug was administered intranasally by inserting drops into the noses of each of a group of six dogs in amounts and three different formulations as listed in Table 1. The intranasal dose per dog was 2 mg in a volume of 0.2 ml. Dogs were anesthetized lightly to avoid sneezing reflex. At each of the indicated times, the animals were checked for emesis. At a given time, the number of dogs having emesis out of the number of dogs in the group is indicated in the table. For example 2/4 in the table indicates that two dogs of a group of four had emesis at a given time. This data was compared to data obtained in a previous study, wherein a group of four dogs were monitored for emesis after the same time intervals after administration by various routes. SL stands for sublingual and SC stands for subcutaneous.

TABLE 1
Comparative Raw Data for Dog Emesis Subsequent to
Intranasal Apomorphine Administration

| | Dose/dog | Incidences of Emesis at Given Times (min) | | | | | | | |
|---|---|---|---|---|---|---|---|---|---|
| Treatment | (mg) | 0 | 5 | 8 | 10 | 15 | 20 | 30 | 60 |
| SC^{a} | 0.4 | — | — | 4/4 | — | 2/4 | — | — | — |
| SL Tablet^{a} | 2 | — | — | — | — | 1/4 | — | 1/4 | — |
| Oral^{a} | 2 | — | — | — | — | — | — | — | — |
| Study 1 | | | | | | | | | |
| Intranasal | 2 | 3/6 | — | — | — | — | 2/6 | — | — |
| 1^{b} | | | | | | | | | |
| Intranasal | 2 | 3/6 | — | — | — | — | 1/6 | 1/6 | — |
| 2^{c} | | | | | | | | | |
| Intranasal | 2 | — | — | — | 4/6 | — | 1/6 | — | — |
| 3^{d} | | | | | | | | | |
| SC^{e} | 1 | — | 1/6 | — | 4/6 | 1/6 | — | — | — |

^{a}= data obtained from study of Example 3 of U.S. Pat. No. 5,994,363
^{b}= Formulation of 1% drug (10 mg/ml), 5% polyoxypropylene/polyoxyethylene block copolymer (PLURONIC F127) and 1% sodium metabisulfite (stabilizer) in water
^{c}= Formulation of 1% drug (10 mg/ml), 15% polyoxypropylene/polyoxyethylene block copolymer (PLURONIC F127) and 1% sodium metabisulfite (stabilizer) in water
^{d}= Formulation of 1% drug (10 mg/ml), 15% polyoxypropylene/polyoxyethylene block copolymer (PLURONIC F127), 0.6% hydroxypropyl methyl cellulose (METHOCEL K100 LV, bioadhesive agent) and 1% sodium metabisulfite (stabilizer) in water
^{e}= Formulation of 0.04% drug (0.4 mg/ml) and 1% sodium metabisulfite in water

Table 2 below shows the analysis of the raw data provided in Table 1 above. Bioavailability is measured relative to subcutaneous administration, which provides 100% bioavailability. Cₘₐₓ is the maximum blood plasma concentration; Tₘₐₓ is the time from dosing until maximum blood serum concentration is obtained; average severity (AS) is calculated as total incidences of emesis over time divided by number of dogs studied, expressed as a percentage. AS/Cₘₐₓ is a measure of severity with respect to maximal concentration of the drug. A higher AS/Cₘₐₓ value indicates that there is a greater proportion of side effects (measured here as emesis) relative to the amount of drug in the subject's system. Moreover, a lower AS/Cₘₐₓ value indicates that there is a lesser proportion of side effects relative to the amount of the drug in the subject's system. Therefore, lower AS/Cₘₐₓ values are desirable. Note also that an AS of 50% in dogs is approximately equivalent to an AS of 5% in humans, due to the much higher sensitivity in dogs than humans.

Table 2 shows that the intranasal administration results in a greatly increased Cₘₐₓ and bioavailability over sublingual administration at the same dosage level. However, contrary to conventional behavior, the increase in severity of side effects is not also proportionally increased. The last column of Table 2 illustrates this point. Therefore, intranasal administration unexpectedly results in a more effective bioavailability than sublingual administration without a proportional increase in adverse side effects.

TABLE 2
Analysis of Comparative Raw Data for Dog Emesis Subsequent to
Intranasal Apomorphine Administration

| | Dose/ | | | | Average | |
|---|---|---|---|---|---|---|
| | dog | Tₘₐₓ | Cₘₐₓ | Bioavailability | Severity | AS/ |
| Treatment | (mg) | (hr) | (ng/ml) | (%) | (%) | Cₘₐₓ |
| SC^{a} | 0.4 | 0.25 | 8.46 | 100 | 150 | 17.7 |
| SL Tablet^{a} | 2 | 0.38 | 7.75 | 13.5 | 50 | 6.5 |
| Oral^{a} | 2 | 0.35 | 0.40 | 3.9 | 0 | 0 |
| Study 1 | | | | | | |
| Intranasal 1^{b} | 2 | 0.17 | 139.2 | 150.8 | 83 | 0.6 |
| Intranasal 2^{c} | 2 | 0.27 | 161.4 | 126.9 | 83 | 0.5 |
| Intranasal 3^{d} | 2 | 0.17 | 1152.6 | 105.8 | 83 | 0.5 |
| SC^{e} | 1 | | | 100 | 100 | |

a = data obtained from study of Example 3 of U.S. Pat. No. 5,994,363
b = Formulation of 1% drug (10 mg/ml), 5% polyoxypropylene/polyoxyethylene block copolymer (PLURONIC F127) and 1% sodium metabisulfite (stabilizer) in water
c = Formulation of 1% drug (10 mg/ml), 15% polyoxypropylene/polyoxyethylene block copolymer (PLURONIC F127) and 1% sodium metabisulfite (stabilizer) in water
d = Formulation of 1% drug (10 mg/ml), 15% polyoxypropylene/polyoxyetbylene block copolymer (PLURONIC F127), 0.6% hydroxypropyl methyl cellulose (METHOCEL K100 LV, bioadhesive agent) and 1% sodium metabisulfite (stabilizer) in water
e = Formulation of 0.04% drug (0.4 mg/ml) and 1% sodium metabisulfite in water

### EXAMPLE 2

The experimental procedure of Example 1 was utilized to obtain information on moderation of undesirable side effects when administration of apomorphine is by inhalation, as compared to the conventional sublingual route. A solution was introduced directly to the dogs' lungs through a hole made in the trachea of each dog, to represent administration of an aerosolized drug which deposits in the lungs. The results of the study are shown in Table 3.

TABLE 3
Comparative Raw Data for Dog Emesis Subsequent to
Apomorphine Administration by Inhalation

| | Dose/dog | Incidences of Emesis at Given Times (min) | | | | | | | |
|---|---|---|---|---|---|---|---|---|---|
| Treatment | (mg) | 0 | 5 | 8 | 10 | 15 | 20 | 30 | 60 |
| SC^{a} | 0.4 | — | — | 4/4 | — | 2/4 | — | — | — |
| SL Tablet^{a} | 2 | — | — | — | — | 1/4 | — | 1/4 | — |
| Oral^{a} | 2 | — | — | — | — | — | — | — | — |
| Study 2 | | | | | | | | | |
| Inhalation | 0.5 | — | 4/5 | — | — | — | — | — | — |
| 1^{b} | | | | | | | | | |
| Inhalation | 1 | 5/5 | — | — | — | — | — | — | — |
| 2^{c} | | | | | | | | | |
| Inhalation | 2 | 5/5 | — | — | — | — | — | — | — |
| 3^{d} | | | | | | | | | |

^{a}= data obtained from study of Example 3 of U.S. Pat. No. 5,994,363
^{b}= Formulation of 0.05% drug (0.5 mg/ml) and 1% sodium metabisulfite (stabilizer) in water; 1 ml per dog
^{c}= Formulation of 0.1% drug (1 mg/ml) and 1% sodium metabisulfite (stabilizer) in water; 1 ml per dog
^{d}= Formulation of 0.2% drug (2 mg/ml) and 1% sodium metabisulfite (stabilizer) in water; 1 ml per dog

Table 4 below shows the analysis of the raw data provided in Table 3 above. The drug administration to the lungs results in a greatly increased bioavailability over sublingual administration at the same, as well as at lower, dosage levels. However, contrary to conventional behavior, the increase in severity of side effects is not also proportionally increased. The last column of Table 4 illustrates this point. Therefore, administration by inhalation results in more effective bioavailability than sublingual administration without a proportional increase in adverse side effects. It is particularly noteworthy that this method of dosage administration allows a dose proportionate increase in Cₘₐₓ, an expected phenomenon, while reducing AS/Cₘₐₓ, an unexpected phenomenon.

TABLE 4
Analysis of Comparative Raw Data for Dog Emesis Subsequent to
Apomorphine Administration by Inhalation

| | | | | Bioavail- | Average | |
|---|---|---|---|---|---|---|
| | Dose/dog | Tₘₐₓ | Cₘₐₓ | ability | Severity | |
| Treatment | (mg) | (hr) | (ng/ml) | (%) | (%) | AS/Cₘₐₓ |
| SC^{a} | 0.4 | 0.25 | 8 46 | 100 | 150 | 17.7 |
| SL Tablet^{a} | 2 | 0.38 | 7.75 | 13.5 | 50 | 6.5 |
| Oral^{a} | 2 | 0.35 | 0.40 | 3.9 | 0 | 0 |
| Study 2 | | | | | | |
| Inhalation | 0.5 | 0.17 | 15.2 | 67.2 | 80 | 5 3 |
| 1^{b} | | | | | | |
| Inhalation | 1 | 0.17 | 31.5 | 62.7 | 100 | 3.2 |
| 2^{c} | | | | | | |
| Inhalation | 2 | 0.17 | 65 1 | 63.9 | 100 | 1.5 |
| 3^{d} | | | | | | |

^{a}= data obtained from study of Example 3 of U.S. Pat. No. 5,994,363
^{b}= Formulation of 0.05% drug (0.5 mg/ml) and 1% sodium metabisulfite (stabilizer) in water; 1 ml per dog
^{c}= Formulation of 0.1% drug (1 mg/ml) and 1% sodium metabisulfite (stabilizer) in water; 1 ml per dog
^{d}= Formulation of 0.2% drug (2 mg/ml) and 1% sodium metabisulfite (stabilizer) in water; 1 ml per dog

### EXAMPLE 3

The experimental procedure of Example 1 was utilized to obtain information on the moderation of undesirable side effects when apomorphine is administered orally by various formulations, as compared to the conventional sublingual route or oral route. Test formulations were introduced directly to the dogs' stomach as a solution through a tube or in capsule form. The results of the study are shown in Table 5.

TABLE 5
Comparative Raw Data for Dog Emesis Subsequent to
Oral Apomorphine Administration

| | | Incidences of Emesis at Given Times | | | | | | | |
|---|---|---|---|---|---|---|---|---|---|
| | Dose/dog | (min) | | | | | | | |
| Treatment | (mg) | 0 | 5 | 8 | 10 | 15 | 20 | 30 | 60 |
| SC^{a} | 0.4 | — | — | 4/4 | — | 2/4 | — | — | — |
| SL Tablet^{a} | 2 | — | — | — | — | 1/4 | — | 1/4 | — |
| Oral^{a} | 2 | — | — | — | — | — | — | — | — |
| Study 3 | | | | | | | | | |
| Oral 1^{b} | 10 | — | — | — | — | — | — | — | 1/5 |
| 10 mg/ml gavage | | | | | | | | | |
| Oral 2^{c} | 20 | 2/5 | — | 3/5 | — | — | — | — | — |
| 20 mg/ml gavage | | | | | | | | | |
| Oral 3^{d} | 10 | — | — | — | — | — | — | — | — |
| capsules | | | | | | | | | |

^{a}= data obtained from study of Example 3 of U.S. Pat. No. 5,994,363
^{b}= Formulation of 1% drug (0.5 g) and 1% (0.5 g) sodium metabisulfite (stabilizer) in water
^{c}= Formulation of 2% drug (1 g) and 1% (0.5 g) sodium metabisulfite (stabilizer) in water
^{d}= Formulation of 10% drug (10 mg) and 90% (90 mg) of Avicel 101 (microcyrstalline cellulose)

Table 6 below shows the analysis of the raw data provided in Table 5 above. The relationship of bioavailability to severity of undesirable side effects can be controlled by varying the formulation for oral administration. Oral formulation 2 results in a higher bioavailability than oral formulation 1, yet oral formulation 2 produces less severe side effects in relationship to bioavailability than oral formulation 1. The last column of Table 6 illustrates this point. Also of note is that different oral formulations produce varying Cₘₐₓ values. The oral formulation 2 resulted in nearly a four-fold higher Cₘₐₓ compared to sublingual tablets without a comparable increase in emesis. Therefore, depending upon the formulation, Cₘₐₓ versus side effects can also be optimized.

TABLE 6
Analysis of Comparative Raw Data for Dog Emesis Subsequent to
Oral Apomorphine Administration

| | | | | Bioavail- | Average | |
|---|---|---|---|---|---|---|
| | Dose/dog | Tₘₐₓ | Cₘₐₓ | ability | Severity | |
| Treatment | (mg) | (hr) | (ng./ml) | (%) | (%) | AS/Cₘₐₓ |
| SC^{a} | 0.4 | 0.25 | 8.46 | 100 | 150 | 17.7 |
| SL Tablet^{a} | 2 | 0.38 | 7.75 | 13.5 | 50 | 6.5 |
| Oral^{a} | 2 | 0.35 | 0.40 | 3.9 | 0 | 0 |
| Study 3 | | | | | | |
| Oral 1^{b} | 10 | 0.13 | 4 21 | 1.83 | 20 | 4.75 |
| 10 mg/ml | | | | | | |
| gavage | | | | | | |
| Oral 2^{c} | 20 | 0.35 | 29 3 | 3.87 | 100 | 3.4 |
| 20 mg/ml | | | | | | |
| gavage | | | | | | |
| Oral 3^{d} | 10 | 0.19 | 1.75 | 1.16 | 0 | 8.6 |
| capsule | | | | | | |

^{a}= data obtained from study of Example 3 of U.S. Pat. No. 5,994,363
^{b}= Formulation of 1% drug (0.5 g) and 1% (0.5 g) sodium metabisulfite (stabilizer) in water
^{c}= Formulation of 2% drug (1 g) and 1% (0.5 g) sodium metabisulfite (stabilizer) in water
^{d}= Formulation of 10% drug (10 mg) and 90% (90 mg) of Avicel 101 (microcyrstalline cellulose)

### EXAMPLE 4

A study was done to determine apomorphine absorption in humans at varying dose levels. Twenty-four men were tested using dosages of 2, 4, 5 and 6 mg sublingual tablets. Plasma samples were obtained from each subject immediately after placing the tablet under the tongue, followed by further sampling at specified time intervals, up to 20 minutes. After 20 minutes under the tongue, the remaining undissolved mass (if any) was discarded. The samples were then assayed using a highly sensitive LC/MS/MS technique. Peak plasma drug levels approximating 0.70, 1.25, 1.70 and 1.91 ng/ml respectively were reported, as indicated in Table 7. In the table, SD stands for standard deviation. These results indicate that apomorphine is absorbed in a dose-proportionate manner (Cₘₐₓ as well as AUC (area under the curve) increased linearly with increase in sublingual tablet dose). Since up to a 6 mg dose delivered via a sublingual tablet has been shown to offer good efficacy and minimal side-effects in humans, plasma drug levels attained following administration of 6 mg apomorphine as a sublingual tablet are meaningful indicators of performance. In other words, plasma drug levels between 0 to 6 ng/ml in humans (obtained with 6 mg tablet), following sublingual administration as a tablet, are meaningful indicators of good efficacy and low side-effects in the treatment of sexual dysfunction. The bioavailability of sublingual tablets in humans, relative to a subcutaneous control, was estimated to be 16-18%.

TABLE 7
Apomorphine Pharmacokinetic Parameters in Humans

| | | 2 mg | 4 mg | 5 mg | | |
|---|---|---|---|---|---|---|
| Parameter | | SL | SL | SL | 6 mg SL | 1 mg SC |
| tₘₐₓ (h) | Mean | 0.74 | 0.72 | 0.68 | 0.66 | 0.34 |
| | SD | 0.30 | 0.32 | 0.21 | 0.32 | 0.17 |
| cₘₐₓ (ng/ml) | Mean | 0.70 | 1.25 | 1.70 | 1.91 | 3.22 |
| | SD | 0.37 | 0.80 | 1.32 | 1.22 | 1.67 |
| AUC_{∞}(ng·h/ml) | Mean | 1.23 | 2.37 | 2.92 | 3.60 | 3.39 |
| | SD | 0.48 | 1.06 | 1.50 | 1.73 | 1.09 |

Clinical experience with 2 to 4 mg sublingual apomorphine tablets in humans has demonstrated about 13% incidence of nausea and 2% incidence of emesis. Any formulation or dosage administration technique which allows drug levels to be attained in the range of 0.25 to 5 ng/ml with less side effects such as emesis can be expected to improve patient compliance, and usefulness of this compound in the treatment of sexual dysfunction. Dogs have been indicated to be much more sensitive to emesis than humans, as has been previously described. Hence, any formulation or dosage which enables drug levels in dogs comparable to that achievable with sublingual tablets without comparable emesis profile is believed to have superior performance in humans. The intranasal, inhalation to the lungs or oral formulations investigated in this work demonstrate that this can be achieved.

Sub-lingual apomorphine tablets have demonstrated approximately 15% relative bioavailability against sub-cutaneous human control in humans as well as in dogs. This suggests that the dog is a good model in representing absorption of apomorphine. Up to 8 mg of apomorphine tablets have been shown to be well tolerated in humans. Assuming a 60 kg human weight and a 10 kg dog weight, an 8 mg human dose compares well with about 1.33 mg apomorphine dose in dogs. For the studies presented here, dosages in the range of 0.5 to 20 mg/dog were investigated to achieve plasma drug levels in dogs comparable to or higher than those achieved with 2 mg sublingual tablets in dogs without comparable side-effects. The intranasal, inhalation to the lungs or oral routes of administration investigated in the above examples demonstrate that this can be achieved.

All references cited are hereby incorporated by reference.

The present invention is illustrated by way of the foregoing description and examples. The foregoing description is intended as a non-limiting illustration, since many variations will become apparent to those skilled in the art in view thereof. It is intended that all such variations within the scope and spirit of the appended claims be embraced thereby.

Changes can be made in the composition, operation and arrangement of the method of the present invention described herein without departing from the concept and scope of the invention as defined in the following claims:

We claim:

1. A method of treating sexual dysfunction in a patient comprising
administering a therapeutically effective amount of apomorphine or a pharmaceutically acceptable salt thereof to said patient;
wherein a concentration of said apomorphine is attained within said patient's plasma of up to 10 nanograms per milliliter;
and wherein said concentration is achieved with less than 15% of patients so treated experiencing emesis;
with the proviso that administration is not sublingual.

2. The method of claim 1 wherein said apomorphine is administered intranasally.

3. The method of claim 2 wherein said apomorphine is administered as a nasal spray, nasal drops, gel, suspension, ointment, cream or powder.

4. The method of claim 1 wherein said apomorphine is administered by oral ingestion.

5. The method of claim 4 wherein said apomorphine is administered as a solution, a suspension, drops, a gel, a tablet, granules, sprinkles, pills, powder, or a capsule.

6. The method of claim 1 wherein said apomorphine is administered by inhalation to the lungs.

7. The method of claim 6 wherein said apomorphine is administered through a metered dose inhaler, dry powder inhaler, nebulized solution or nebulized suspension.

8. The method of claim 1 wherein said sexual dysfunction is erectile dysfunction.

9. The method of claim 1 wherein said patient is female.

10. The method of claim 1 wherein said concentration of apomorphine is from about 0.1 to about 7 ng/ml in said patient's plasma.

11. The method of claim 1 wherein said concentration of apomorphine is from about 0.5 to about 5 ng/ml in said patient's plasma.

12. A method of treating sexual dysfunction in a patient comprising
administering a therapeutically effective amount of apomorphine or a pharmaceutically acceptable salt thereof to said patient intranasally, by inhalation to the lungs or by oral ingestion;
wherein a concentration of said apomorphine is attained within said patient's plasma of up to 10 nanograms per milliliter;
and wherein said concentration is achieved with less than 15% of patients so treated experiencing emesis.

13. The method of claim 12 wherein said apomorphine is administered intranasally as a nasal spray, nasal drops, gel, suspension, ointment, cream or powder.

14. The method of claim 12 wherein said apomorphine is administered by oral ingestion as a solution, a suspension, drops, a gel, a tablet, pills, powder, granules, sprinkles or a capsule.

15. The method of claim 12 wherein said apomorphine is administered by inhalation to the lungs by a metered dose inhaler, dry powder inhaler, nebulized solution or nebulized suspension.

16. The method of claim 12 wherein said sexual dysfunction is erectile dysfunction.

17. The method of claim 12 wherein said patient is female.

18. The method of claim 12 wherein said concentration of apomorphine is from about 0.1 to about 7 ng/ml in said patient's plasma.

19. The method of claim 12 wherein said concentration of apomorphine is from about 0.5 to about 5 ng/ml in said patient's plasma.
